(19) United States
(12) Patent Application Publication
(10) Pub. No.: US 2014/0309716 A1
(43) Pub. Date: Oct. 16, 2014

(54) METHOD FOR OPERATING A DIRT STOP SURFACE, DIRT STOP SURFACE UNIT AND DIRT STOP SURFACE ASSEMBLED THEREFROM

(71) Applicant: BODYFLOW INTERNATIONAL PTY LTD., Port Melbourne, Victoria 3207 (AU)

(72) Inventor: Joe Di Santo, Blackburn North (AU)

(73) Assignee: BODYFLOW INTERNATIONAL PTY LTD

(21) Appl. No.: 14/358,245

(22) PCT Filed: Mar. 16, 2012
(86) PCT No.: PCT/AU2012/000267

§ 371 Date: May 15, 2014

(30) Foreign Application Priority Data
Nov. 16, 2011 (AU) 2011904779

(51) Int. Cl. A61N 1/36

(52) U.S. Cl. CPC A61N 1/36014
USPC 607/72; 607/115

(57) ABSTRACT

The invention provides a method of reducing an expected length of hospital stay of a patient during a recovery period after surgery, comprising repeated sessions of applying electrical stimulation (W, I) through one or more pairs of electrodes contacting a skin surface in the vicinity of the surgical site.

### FIELD

The present invention relates to recovery from surgery, particularly to methods for reducing a length of hospital stay after surgery.

### BACKGROUND

Most types of surgery typically involve a period of lymphoedema (swelling partly caused by the accumulation of lymph) associated with the natural processes of tissue recovery. Minimising oedema after surgery by the use of ice, compression bandages and anti-inflammatory drugs is a known adjunct to recovery strategies, in many post-surgical situations but particularly in relation to orthopaedic surgery.

There is a need to provide improved rate of recovery from surgery, as this impacts greatly on the success and total cost of treatment. One of the greatest costs associated with surgery is length of hospital stay.

The inventors have conceived the idea that a method of accelerating recovery that has been previously mainly used in the context of sporting injuries may also be applicable to reducing an average hospital stay of patients after surgery, in particular for orthopaedic surgery but conceptually applicable to other forms of surgery.

### SUMMARY OF THE INVENTION

According to a first broad aspect of the invention there is provided a method of reducing an expected length of hospital stay of a patient during a recovery period after surgery, comprising repeated sessions of applying electrical stimulation through one or more pairs of electrodes contacting a skin surface in the vicinity of the surgical site.

In one embodiment, the surgery is an orthopaedic surgery such as knee surgery.

In one embodiment, the electrical stimulation has a stimulation time profile comprising short pulses of current having a pulse width substantially shorter than an interval between the pulses. The pulse width may be less than 12 milliseconds, or less than 8 milliseconds, or less than 3 milliseconds, or about 2 milliseconds.

In one embodiment, the interval between the pulses is greater than 300 milliseconds and less than 900 milliseconds. In another embodiment, the interval between the pulses is greater than 400 milliseconds and less than 700 milliseconds. In another embodiment, the interval between the pulses is about 500 milliseconds. In another embodiment, the interval between the pulses is about 650 milliseconds.

In one embodiment, an instantaneous maximum current delivered during the pulses is less than 200 mA. In another embodiment, an instantaneous maximum current delivered during the pulses is less than 80 mA. In another embodiment, an instantaneous maximum current delivered during the pulses is less than 20 mA. Typically, an instantaneous maximum current delivered during the pulses is greater than 1 mA.

In one embodiment, the pulses comprise a first series of multiple pulses of a first polarity interspersed with a second series of multiple pulses of an opposite second polarity. There may be less than 20, 10 or about 5 pulses in either of the series of pulses.

In one embodiment, most of the sessions comprise a total period of application of less than 30 minutes and greater than 5 minutes. In another embodiment, most of the sessions comprise a total period of application of about 20 minutes.

In one embodiment, the sessions are repeated a multiple number of times per day during a period of hospital stay after the surgery.

In one embodiment, sessions are repeated a multiple number of times per day while recovering at home after the surgery.

According to a second broad aspect of the invention there is provided use of electricity applied through one or more pairs of electrodes contacting a skin surface in the vicinity of a surgical site in a patient, so as to reduce an expected hospital stay of the patient during a recovery period after surgery.

### BRIEF DESCRIPTION OF DRAWINGS

FIG. 1 is a time profile of each pulse used in one embodiment of the invention;

FIG. 2 is a time profile showing series of pulses used in one embodiment of the invention.

### DETAILED DESCRIPTION OF EMBODIMENTS

An embodiment of the current invention will now be described.

In this example, the device for delivering the electrical stimulation is provided by the Bodyflow® models CX1 and P2Ch, for in-hospital post-operative treatment and post-discharge, self-administered applications, respectively. Both Bodyflow units have been approved for use in Australia by the Therapeutic Goods Association (TGA).

The Bodyflow® units are marketed by Bodyflow International Pty Ltd (Victoria, Australia; www.bodyflowinternational.com). The characteristic electrical pulses produced by these units comprise short pulses in the millisecond range separated by gaps in the second range. These waveforms have previously been shown to reduce oedema and have other therapeutic benefits such as improved blood circulation. The Bodyflow CX1 unit can work in two modes, called “Standard”, which has a pulse width of 6 ms and a time between pulses of 658 ms (1.52 Hz), and “light”, which has a pulse width of 6 ms and a time between pulses of 580 ms (1.72 Hz). The Bodyflow P2Ch unit also can work in two modes, called “Standard”, which has a pulse width of 2 ms and a time between pulses of 500 ms (2.0 Hz), and “light”, which has a pulse width of 2 ms and a time between pulses of 580 ms (1.72 Hz). Early research on the range of useful pulse widths, frequencies and polarities for an earlier device, and adopted and adapted herein was reported in US Patent application publication number 2006/0064129.

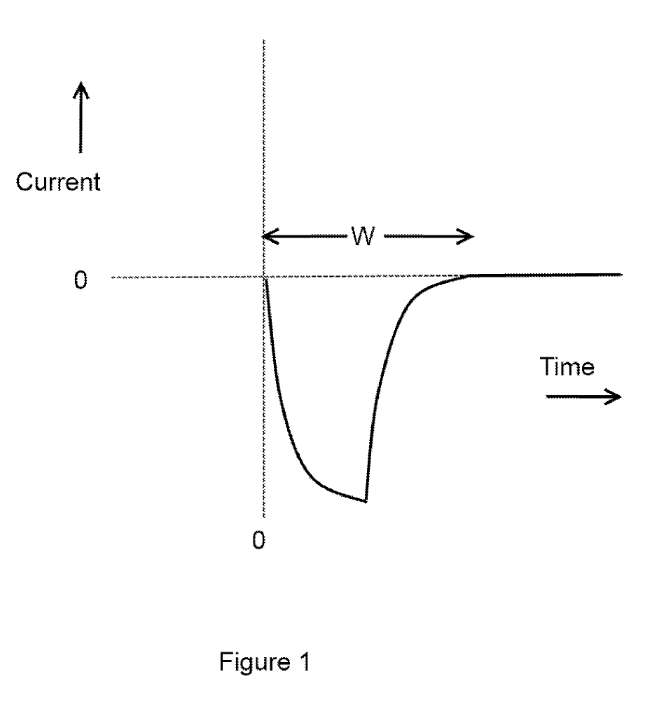

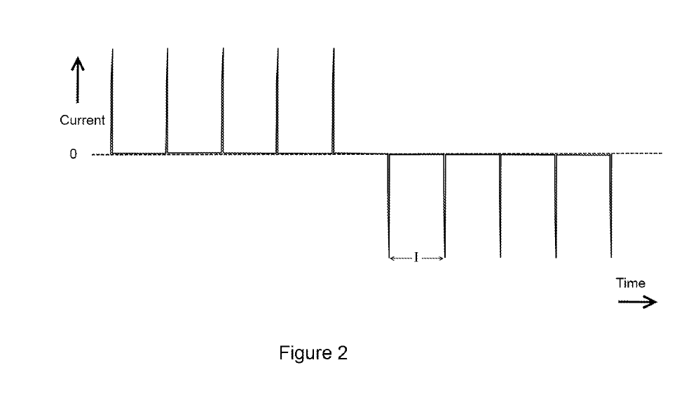

Referring now to FIG. 1, the time profile of each pulse in the Bodyflow units is shown, with pulse width W marked. Referring to FIG. 2, an expanded time profile is shown of the pulse train, which appears in interspersed series of pulses with opposite polarities, each pulse separated by an interval I, 5 pulses in each series. The Bodyflow units have an intensity control for the delivered maximum current which is adjustable from a delivered maximum current of 0 mA up to 75 mA. In practice, the appropriate level depends on the nature of the tissue, the distance between the electrodes and the desired effect. Typically, in use, the intensity control is adjusted upwards until there is a slight visible muscular twitch in response to each pulse and either maintained at around this level or slightly below. For the application contemplated here, the maximum current typically greater than 1 mA and is usually found to be around 5 to 15 mA. Treatment sessions typically last around 20 minutes and may involve one or two pairs of electrodes.

The current example relates to total knee replacement surgery. Total knee replacement in some form has been practiced for over 50 years. The complexities of the knee joint only began to be understood 30 years ago. Because of this, total knee replacement initially was not as successful as Sir John Charnley's artificial hip. However, dramatic advancements in the knowledge of knee mechanics have led to design modifications that appear to be durable. Significant advances have occurred in the type and quality of the metals, polyethylene, and, more recently, ceramics used in the prosthesis manufacturing process, leading to improved longevity. As with most techniques in modern medicine, more and more patients are receiving the benefits of total knee replacement.

There are some complications of Total Knee Replacements (TKR) that can be aggravated by a prolonged hospital stay, including infection and post-operative peripheral oedema. Oedema may result in increased pain levels, and an extended period of decreased activity. Decreased activity, and in particular decreased walking, contributes to the level of oedema, so early mobilization of the patient is important.

It is therefore important to keep the post-operation hospitalization as short as possible. As a result of shortened hospital stay, the costs for the patient's hospitalization will be reduced firstly as a direct effect of the shorter stay, and secondly by preventing prolonged hospitalizations due to above mentioned complications.

The aim is therefore to reduce the expected hospital stay (the time between the operation and the patient's discharge) following total knee replacement surgery. This can be measured in a clinical trial by evaluating a difference in the average hospital stay between Bodyflow treated groups and suitable control groups, such as sham Bodyflow-treated groups and no-Bodyflow groups. The treatment is added to the standard practice of care.

In this example the Bodyflow models CX1 and P2Ch are used, for in-hospital post-operative treatment and post-discharge, self-administered applications, respectively. Both Bodyflow units have been approved for use in Australia by the Therapeutic Goods Association (TGA).

The Bodyflow therapy in this example consists of approximately 4-5 days of in-hospital treatment for 4 periods of 20 minutes a day (preferably using the “standard” waveform setting), followed by a treatment that is delivered by the patient at home for up to 14 days post-surgery for 3 sessions of 20 minutes each day.

The in-hospital unit consists of a stimulator, to which 4 suction cups with stimulating electrodes are connected. Two cups are each positioned above and below the knee, medially and laterally. Electrodes are held in position with suction cups, also connected to the stimulator unit. Suction cups and electrodes can be used multiple times by the same patient, and are cleaned and disinfected in-between treatments.

The unit the patient is discharged with is a small portable handheld stimulator that is connected to 4 electrodes, to be positioned above and below the knee, medially and laterally. These electrodes are adhesive electrodes that can be used multiple times by the same patient, until the adhesive and conductive properties of the electrode are no longer effective.

The therapy is well tolerated, and in many cases hardly perceptible to the patient. There are no reported side effects of this therapy and it does not need to be delivered by a medically trained specialist provided contraindications have been ruled out.

For the first 1-2 days after surgery, preferably after removal of the drainage tube, a lower maximum intensity is used, by increasing the intensity until a slight muscle twitch is evident, and then reducing the intensity slightly. For the remainder of the treatment days, the intensity at which muscle twitch is visible is maintained.

Persons skilled in the art will also appreciate that many variations may be made to the invention without departing from the scope of the invention.

For example, while the example is detailed in relation to total knee replacement, the broadest aspects of the invention extend to recovery from all other forms of orthopaedic and non-orthopaedic surgery unless specifically excluded for safety or practical reasons.

Further, while the example is given in relation to a particular form of electrical stimulation provided by the Bodyflow that is known to reduce lymphoedema, success of the invention may not necessarily require a lymphoedema-reducing form of stimulation. Reduction in hospital stay after surgery may be provided by other forms of electrical stimulation such as interferential and TENS, that have been associated with accelerated healing in the physiotherapy context but have also not previously been considered to be useful in reducing hospital stay. Accordingly, the broadest aspect of the invention extends to the use of any form of electrical stimulation on the skin surface.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

1. A method of reducing an expected length of hospital stay of a patient during a recovery period after surgery, comprising repeated sessions of applying electrical stimulation through one or more pairs of electrodes contacting a skin surface in the vicinity of the surgical site.

2. A method as claimed in claim 1, wherein the surgery is an orthopaedic surgery.

3. A method as claimed in claim 2, wherein the orthopaedic surgery is a knee surgery.

4. A method as claimed in claim 1, wherein the electrical stimulation has a stimulation time profile comprising short pulses of current having a pulse width substantially shorter than an interval between the pulses.

5. A method as claimed in claim 4, wherein the pulse width is less than 12 milliseconds.

6. A method as claimed in claim 4, wherein the pulse width is less than 8 milliseconds.

7. A method as claimed in claim 4, wherein the pulse width is less than 3 milliseconds.

8. A method as claimed in claim 4, wherein the pulse width is about 2 milliseconds.

9. A method as claimed in claim 4, wherein the interval between the pulses is greater than 300 milliseconds and less than 900 milliseconds.

10. A method as claimed in claim 9, wherein the interval between the pulses is greater than 400 milliseconds and less than 700 milliseconds.

11. A method as claimed in claim 4 wherein an instantaneous maximum current delivered during the pulses is less than 200 mA.

12. A method as claimed in claim 4 wherein an instantaneous maximum current delivered during the pulses is less than 80 mA.

13. A method as claimed in claim 4 wherein an instantaneous maximum current delivered during the pulses is less than 20 mA.

14. A method as claimed in claim 4 wherein the pulses comprise a first series of multiple pulses of a first polarity interspersed with a second series of multiple pulses of an opposite second polarity.

15. A method as claimed in claim 14 wherein there are less than 20 pulses in either of the series of pulses.

16. A method as claimed in claim 14 wherein there are about 10 pulses in either of the series of pulses.

17. A method as claimed in claim 14 wherein there are about 5 pulses in either of the series of pulses.

18. A method as claimed in claim 1 wherein most of the sessions comprise a total period of application of less than 30 minutes and greater than 5 minutes.

19. A method as claimed in claim 1 wherein most of the sessions comprise a total period of application of about 20 minutes.

20. A method as claimed in claim 1 wherein the sessions are repeated a multiple number of times per day during a period of hospital stay after the surgery.

21. A method as claimed in claim 20 wherein the sessions are further repeated a multiple number of times per day while recovering at home after the surgery.

22. Use of electricity applied through one or more pairs of electrodes contacting a skin surface in the vicinity of a surgical site in a patient, so as to reduce an expected hospital stay of the patient during a recovery period after surgery.
